(19) United States
(12) Patent Application Publication
(10) Pub. No.: US 2026/0124125 A1
(43) Pub. Date: May 7, 2026

(54) TOOTHPASTES DOPED WITH NANO-HYDROXYAPATITE AND ESSENTIAL OILS

(71) Applicant: Imam Abdulrahman Bin Faisal University, Dammam (SA)

(72) Inventors: Maria IBRAHIM, Dammam (SA); Jehan ALHUMAID, Dammam (SA); Amal ALZAHRANI, Dammam (SA); Banin ALFARDAN, Dammam (SA); Shahd ALGHAMDI, Dammam (SA)

(73) Assignee: Imam Abdulrahman Bin Faisal University, Dammam (SA)

(21) Appl. No.: 18/940,399

(22) Filed: Nov. 7, 2024

(51) Int. Cl. A61K 8/24; A61K 8/21; A61K 8/92; A61Q 11/00

(52) U.S. Cl. CPC A61K 8/24; A61K 8/21; A61K 8/922; A61Q 11/00; A61K 2800/612; A61K 2800/651

(57) ABSTRACT

A toothpaste formulation having re-mineralization and antibacterial properties includes nano-hydroxyapatites in an amount of about 8 wt. % to about 12 wt. % and essential oils in an amount of about 2.8 wt. % to about 3.2 wt. % based on a total weight of the toothpaste formulation The toothpaste formulation further includes a fluoride toothpaste in an amount of about 85 wt. % to about 89 wt. % based on a total weight of the toothpaste formulation. The formulations can reverse carious lesions at the initial stage and can prevent future caries formation. Moreover, the formulations are cost effective and have reduced adverse effects.

### BACKGROUND

### Technical Field

The present disclosure is directed to toothpaste formulations, particularly toothpaste formulations including a combination of nano-hydroxyapatite, fluoride, and essential oils, which attribute re-mineralizing and antibacterial properties to the formulations.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Toothpaste is a commonly used product for maintaining oral hygiene and the health of the gums and the teeth. Various ingredients such as abrasives, binding agents, humectants, surfactants, and fluorides are typically added to toothpaste formulations to impart better cleansing and physical properties. Many toothpaste formulations contain fluoride which helps to protect the enamel layer present on the teeth and also possess anti-caries properties. Additionally, various other ingredients, each having a specific function, are also added to the toothpaste formulations. Such ingredients include flavoring agents, coloring agents, anti-bacterial agents, sweetening agents, and the like. In addition to having desirable physical properties such as easy flow and smooth texture, toothpaste formulations with medicinal effects and repairing functions are preferred. Over time, dental erosion occurs, leading to enamel loss and rendering the teeth hypersensitive to temperature, texture, or taste of food while chewing or drinking. Dental erosions also result in tooth caries and cavities. In this regard, various toothpaste formulations that help in preventing and curing dental erosions are being developed. Some of these include components that are also present in the human teeth, one such component being hydroxyapatite. Hydroxyapatites form an integral part of the mineral composition of human teeth and are also used in toothpaste formulations for their remineralization effects. These hydroxyapatites are prepared at the nano level to form nanohydroxyapatites (nHA) that closely resemble the structure and composition of tooth enamel and, hence, are a preferable choice for tooth remineralization and preventing and curing tooth sensitivity and decay. Many toothpaste formulations having nanohydroxyapatite are currently being launched and used.

Generally, toothpastes also include anti-bacterial agents to prevent dental caries and infections. These anti-bacterial agents are mostly chemical in nature. One such agent is triclosan, which is commonly used in toothpastes. Other commonly used anti-bacterial agents include chlorhexidine, metal salts, and quaternary ammonium compounds such as benzalkonium chloride. To avoid side effects, herbal and chemical-free anti-bacterial agents are currently added to toothpaste. Compounds derived from plants are preferred. Such compounds may be phytochemicals containing saponins, tannins, phenolic compounds, flavonoids and the likes. Essential oils derived from plants are also included in toothpastes. A recent trend in toothpaste research is towards developing compositions which have fewer chemicals and no adverse effects.

US20210378945A1 describes a toothpaste including nano-hydroxyapatite in an amount of 3-40 % by mass of the toothpaste and tea tree oil in an amount of 0.10-1.0% by mass of the toothpaste, aloe in an amount of 5.0-37.0% by mass of the toothpaste, xylitol in an amount of 5.0-40.0% by mass of the toothpaste, silicon dioxide in an amount of 3.0-12.0% by mass of the toothpaste.

US20170189287A1 describes a tooth cleaning composition including a wax and a calcium phosphate. The calcium phosphate is present in the form of hydroxyapatite in a content of 0.001 to 33% by mass and the wax is present in a content of 0.0001 to 30% by mass.

US20230114022A1 describes a toothpaste including nano-hydroxyapatite in a range of 0.025% w/w to 70% w/w, coconut oil, tea tree oil and cinnamon bark oil in an amount of 0.05-10% v/w.

CN115624499A describes a whitening anti-allergic toothpaste including 4-12 % of nano-hydroxyapatite, 0.5-5% of chitosan oligosaccharide, 0.1-0.5% of lactoferrin, 0.01-0.5% of biological lysozyme, 0.5-3% of cinnamon extract, 0.5-5% of honeysuckle extract, 0.1-0.5% of dendrobium officinale extract, 0.1-0.5% of propolis, 0.5-3% of polyethylene glycol, 0.3-0.6% of xanthan gum, 0.1-0.5% of probiotics, 0.1-0.5% of phytic acid, 0.1-0.5% of xylitol and 0.05-0% of triclosan 15%, purified water margin.

CN105640788A describes a hydroxyapatite toothpaste including nanohydroxyapatite in a mass content of not more than 20% and fluoride in a mass content of not more than 8.0 wt. %.

Each of the aforementioned patent literature suffers from one or more drawbacks hindering their adoption, such as a higher content of nanohydroxyapatite, lack of fluorides, essential oils and other effective anti-bacterial agents. Additionally, most of the existing toothpaste formulations contain chemical ingredients which may appear beneficial for their antimicrobial and dental repairing activities but have adverse effects when used on a regular basis. Accordingly, it is an object of the present disclosure to provide toothpaste formulations with lower chemical content and reduced side effects. Another object of the present disclosure is to provide toothpaste formulations, including essential oils, fluoride, and nanohydroxyapatite, which have remineralization and anti-bacterial properties.

### SUMMARY

In an exemplary embodiment, a toothpaste formulation is described. The toothpaste formulation includes a nano-hydroxyapatite in an amount of about 8 wt. % to about 12 wt. %, an essential oil in an amount of about 2.8 wt. % to about 3.2 wt. %, wherein the essential oil is selected from the group consisting of tea tree oil, coconut oil, cinnamon oil and combinations thereof, and the balance fluoride toothpaste, where wt. % is based on a total weight of the toothpaste formulation. A first portion of the essential oil is dissolved in the fluoride toothpaste and a second portion of the essential oil is bonded or adsorbed in the nano-hydroxyapatite.

In another exemplary embodiment, a toothpaste formulation consisting of a nano-hydroxyapatite in an amount of about 8 wt. % to about 12 wt. %, an essential oil in an amount of about 2.8 wt. % to about 3.2 wt. %, where the essential oil is selected from the group consisting of tea tree oil, coconut oil, cinnamon oil and combinations thereof, and the balance fluoride toothpaste, wherein wt. % is based on a total weight of the toothpaste formulation. A first portion of the essential oil is dissolved in the fluoride toothpaste, and a second portion of the essential oil is bonded or adsorbed in the nano-hydroxyapatite.

In yet another exemplary embodiment, a method of formulating an antibacterial and remineralizing toothpaste is described. The method includes mixing an essential oil in an amount of about 2.8 wt. % to about 3.2 wt. %, where the essential oil is selected from the group consisting of tea tree oil, coconut oil, cinnamon oil and combinations thereof, with a nano-hydroxyapatite in an amount of about 8 wt. % to about 12 wt. % to form a first mixture, and mixing a fluoride toothpaste in an amount of about 85 wt. % to about 89 wt. % into the first mixture to form a second mixture, wherein a first portion of the essential oil is dissolved in the fluoride toothpaste and a second portion of the essential oil is bonded or adsorbed in the nano-hydroxyapatite, and sterilizing the second mixture under ultraviolet lighting.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is a schematic flowchart of a method of preparing a toothpaste formulation, according to certain embodiments.

FIG. 2 depicts various experimental toothpaste formulations tested by three different study groups, according to certain embodiments.

FIG. 3 depicts a process for assessing re-mineralization properties of the toothpaste formulation, according to certain embodiments.

FIG. 4 depicts a process for assessing anti-bacterial properties of the toothpaste formulation, according to certain embodiments.

FIG. 5 shows a graph representing surface microhardness percent difference for essential oils alone, according to certain embodiments.

FIG. 6 shows a graph representing surface microhardness percent difference for essential oils with nano-hydroxyapatite, according to certain embodiments.

FIG. 7 shows a graph representing surface microhardness percent difference for coconut oil, according to certain embodiments.

FIG. 8 shows a graph representing surface microhardness percent difference for cinnamon oil, according to certain embodiments.

FIG. 9 shows a graph representing surface microhardness percent difference for tea tree oil, according to certain embodiments.

FIG. 10 shows a graph representing colony forming units counts for all experimental toothpastes, according to certain embodiments.

FIG. 11 shows a graph representing metabolic activity for all experimental toothpastes, according to certain embodiments.

FIG. 12 shows a graph representing pH measurements (mean 50, n=3) for all experimental toothpastes, according to certain embodiments.

### DETAILED DESCRIPTION

In the drawings, like reference numerals designate identical or corresponding parts throughout the several views. Further, as used herein, the words “a”, “an” and the like generally carry a meaning of “one or more”, unless stated otherwise.

Furthermore, the terms “approximately,” “approximate”, “about” and similar terms generally refer to ranges that include the identified value within a margin of 20%, 10%, or preferably 5%, and any values therebetween.

A weight percent of a component, unless specifically stated to the contrary, is based on the total weight of the formulation or composition in which the component is included. For example, if a particular element or component in a composition or article is said to have 5 wt. %, it is understood that this percentage is in relation to a total compositional percentage of 100%.

Aspects of this disclosure are directed to a toothpaste formulation having excellent re-mineralizing and anti-bacterial properties imparted by natural ingredients. More particularly, the present formulation serves as a method for reversing initial carious lesions and preventing future caries formation due to its anti-bacterial properties.

According to an aspect of the present disclosure, the toothpaste formulation includes a fluoride, a nanohydroxyapatite and at least one essential oil, wherein all the ingredients act to provide effective cleansing and prevent dental caries. Fluoride forms a preventive layer on the tooth enamel and protects it from acids that may be released by microorganisms such as bacteria. Nano-hydroxyapatite helps in remineralization and enamel repair, and essential oils prevent the building of plaques and tartar on the teeth due to their anti-bacterial properties.

Hydroxyapatites are composed of calcium and phosphorus. Due to their composition being similar to human bones and teeth, hydroxyapatites are often preferred for dental repair, including re-mineralizing teeth. Nano-hydroxyapatites are hydroxyapatites that have particle sizes on a nanoscale. Since nanoparticles provide better bioactivity and are resorbed readily, the nano-hydroxyapatites provide more efficient formulations in terms of dental restoration and dental care. In one aspect, the toothpaste formulation includes a nano-hydroxyapatite in an amount of about 8 wt. % to about 12 wt. % based on the total weight of the toothpaste formulation. In some aspects, the toothpaste formulation includes a nano-hydroxyapatite in an amount of about 8.5 wt. % to about 11.5 wt. %, preferably about 9 wt. % to about 11 wt. %, preferably about 9.5 wt. % to about 10.5 wt. %, preferably about 10 wt. % based on the total weight of the toothpaste formulation.

The toothpaste formulation includes a fluoride toothpaste in an amount of about 85 wt. % to 89 wt. % based on the total weight of the toothpaste formulation. In an aspect, the fluoride toothpaste is present in the toothpaste formulation in an amount of about 85.5 wt. % to 88.5 wt. % based on the total weight of the toothpaste formulation. In some aspects, the fluoride toothpaste is present in the toothpaste formulation in an amount of about 86 wt. % to 88 wt. % based on the total weight of the toothpaste formulation, preferably about 86.5 wt. % to 87.5 wt. % based on the total weight of the toothpaste formulation, preferably about 87 wt. % based on the total weight of the toothpaste formulation. In another aspect, the fluoride toothpaste is present in the toothpaste formulation in an amount of about 87 wt. % to 88 wt. % based on the total weight of the toothpaste formulation. The preferred amount for the fluoride toothpaste in the formulation may be about 88 wt. % to 89 wt. % based on the total weight of the toothpaste formulation.

The fluoride toothpaste may include sodium fluoride, stannous fluoride, or sodium monofluorophosphate as the fluoride component. According to the disclosure, any one of sodium fluoride, stannous fluoride, or sodium monofluorophosphate may be present in an amount of about 0.10 to 0.15 wt. %. In one aspect, the fluoride toothpaste in the present formulation includes sodium monofluorophosphate in an amount of about 0.10 to 0.15 wt. %. In another aspect, the fluoride toothpaste in the present formulation includes sodium fluoride in an amount of about 0.10 to 0.15 wt. %. According to the present disclosure, the fluoride toothpaste includes sodium fluoride in an amount of about 0.11 wt. %, preferably about 0.12 wt. %, preferably about 0.13 wt. %, preferably about 0.14 wt. %. The preferred amount of sodium fluoride in the fluoride toothpaste may be about 0.15 wt. %.

The fluoride toothpaste further includes humectants, abrasives, thickening agents, water, and surfactants. Humectants are hygroscopic in nature and are added to various food products, medicines, and cosmetics to retain moisture and prevent the products from drying out. Suitable examples of humectants include, but are not limited to, glycerin, sorbitol, propylene glycol, dipropylene glycol, diglycerol, triacetin, mineral oil, polyethylene glycol (preferably, PEG-400), alkane diols like butane diol and hexanediol, ethanol, pentylene glycol, or a mixture there of.

According to the present disclosure, humectants may be selected from glycerin, polyols such as sorbitol, glycerol, xylitol, polyethylene glycol and other glycols. Humectants may be present in the fluoride toothpaste in an amount of about 80 wt. % to 90 wt. % based on the total weight of the fluoride toothpaste, preferably in an amount of about 85 wt. % to 88 wt. % based on the total weight of the fluoride toothpaste.

Abrasives are added to toothpastes for removing tartar and dental plaques from the teeth. Suitable abrasives include hydrated silica and alumina, carbonates such as calcium and magnesium carbonates, silicates, perlites, calcium pyrophosphate or sodium metaphosphate. In one aspect, the amount of abrasive may vary from 5 wt. % to 15 wt. % based on a total weight of the fluoride toothpaste.

Thickening agents or binders may be added to the fluoride toothpastes in an amount of about 0.5 wt. % to 3 wt. % for desirable consistency. Various thickening agents may be added, such as gelatin, xanthan gums, carboxyvinyl polymers, sodium alginates etc.

The fluoride toothpaste may include conventional surfactants in an amount of about 2 wt. % to 5 wt. % to promote foaming. The surfactants may be selected from a group including sodium lauryl sulphate, sodium dodecyl sulfate and sodium cocoyl glutamate. Non-ionic, zwitterionic or amphoteric surfactants may also be used. Certain other examples of surfactants that can be used, include, but are not limited to, sodium, magnesium, ammonium or ethanolamine salts of C₈ to C₁₈ alkyl sulphates (for example sodium lauryl sulfate), ethoxylated alkyl sulfate anionic surfactant (such as sodium lauryl ether sulfate), C₈ to C₁₈ alkyl sulphosuccinates (for example dioctyl sodium sulphosuccinate), C₈ to C₁₈ alkyl sulphoacetates (such as sodium lauryl sulphoacetate), C₈ to C₁₈ alkyl sarcosinates (such as sodium lauryl sarcosinate), C₈ to C₁₈ alkyl phosphates (which can optionally comprise up to 10 ethylene oxide and/or propylene oxide units) and sulphated monoglycerides, preferably the anionic surfactant is sodium lauryl sulfate. Typical amphoteric surfactants include, for example, alkyl amine oxides, alkyl betaines, alkyl amidopropyl betaines, alkyl sulphobetaines (sultaines), alkyl glycinates, alkyl carboxyglycinates, alkyl amphoacetates, alkyl amphopropionates, alkylamphoglycinates, alkyl amidopropyl hydroxysultaines, acyl taurates and acyl glutamates, wherein the alkyl and acyl groups have from 8 to 19 carbon atoms, preferably the amphoteric surfactant is cocamidopropyl betaine.

In one aspect, distilled water is added to the fluoride toothpaste in an amount of about 1 to 7 wt. % for a smooth outflow of the toothpaste from the dispenser. Water may also be added for proper dissolution of the ingredients of the formulation. The fluoride toothpaste may also include conventional sweetening agents in small amounts of about 0.1 wt. % to 1 wt. % to enhance the taste or flavor of the toothpaste formulation.

According to the present disclosure, the toothpaste formulations include essential oils for their anti-microbial properties and their characteristic flavor. Essential oils are oils derived from certain plants and have specific fragrance. Many of the essential oils have medicinal and healing effects and are found in aromatherapy, air fresheners, food flavoring agents, and certain products to provide a particular fragrance. Commonly used essential oils include peppermint, lavender, tea tree oil, clove oil, and eucalyptus oil. In one aspect, the toothpaste formulation includes essential oil in an amount of about 2.8 wt. % to about 3.2 wt. % based on the total weight of the toothpaste formulation. In some aspects, the toothpaste formulation includes an essential oil in an amount of about 2.9 wt. %, most preferably about 3 wt. %, preferably about 3.1 wt. %, preferably about 3.2 wt. % based on the total weight of the toothpaste formulation. The preferred amount of essential oil may be about 3 wt. % based on the total weight of the toothpaste formulation.

In some aspects, the formulations include at least one essential oil selected from tea tree oil, coconut oil, and cinnamon oil. In an aspect, the toothpaste formulations may include nanohydroxyapatite, tea tree oil as the essential oil, and fluoride toothpaste wherein the nano-hydroxyapatite may be present in an amount of about 10 wt. %, tea tree oil may be present in an amount of about 3 wt. %, and the fluoride toothpaste may be present in an amount of about 87 wt. %.

In another aspect, the toothpaste formulations, including nanohydroxyapatite and fluoride toothpaste, may include coconut oil as the essential oil. In such compositions, the nano-hydroxyapatite may be present in an amount of about 10 wt. %, the coconut oil may be present in an amount of about 3 wt. %, and the fluoride toothpaste is present in an amount of about 87 wt. %.

In some aspects, the toothpaste formulations include cinnamon oil as the essential oil. In such cases, the formulations include nano-hydroxyapatite in an amount of about 10 wt. %, cinnamon oil in an amount of about 3 wt. %, and the fluoride toothpaste in an amount of about 87 wt. %.

The toothpaste formulation may include a combination of two essential oils selected from tea tree oil, coconut oil and cinnamon oil. In an example, the toothpaste formulations may include a combination of tea tree oil and coconut oil. Such formulations include nano-hydroxyapatite in an amount of about 8 wt. % to about 12 wt. %, tea tree oil in an amount of about 1.5 wt. % and coconut oil in an amount of about 1.5 wt. %, and the fluoride toothpaste in an amount of about 85 wt. % to about 89 wt. %.

In some aspects, toothpaste formulations may include a combination of tea tree oil and cinnamon oil. Such formulations include nano-hydroxyapatite in an amount of about 8 wt. % to about 12 wt. %, tea tree oil in an amount of about 1.5 wt. %, cinnamon oil in an amount of about 1.5 wt. %, and fluoride toothpaste in an amount of about 85 wt. % to about 89 wt. %.

In some aspects, the toothpaste formulation may include a combination of cinnamon oil and coconut oil, wherein the formulations include nano-hydroxyapatite in an amount of about 8 wt. % to about 12 wt. %, cinnamon oil in an amount of about 1.5 wt. %, coconut oil in an amount of about 1.5 wt. %, and fluoride toothpaste in an amount of about 85 wt. % to about 89 wt. %.

Toothpaste formulations including a combination of three essential oils selected from a group consisting of tea tree oil, coconut oil and cinnamon oil also form a part of the present disclosure. Such formulations may include nanohydroxyapatite in an amount of about 8 wt. % to about 12 wt. %, a combination of tea tree oil in an amount of about 1.0 wt. %, cinnamon oil in an amount of about 1.0 wt. % and coconut oil in an amount of about 1.0 wt. %, and the fluoride toothpaste in an amount in a range of about 85 wt. % to about 89 wt. %. Some of these formulations may include nanohydroxyapatite in an amount of about 8 wt. %, a combination of tea tree oil in an amount of about 1.0 wt. %, cinnamon oil in an amount of about 1.0 wt. % and coconut oil in an amount of about 1.0 wt. %, and the fluoride toothpaste in an amount of about 89 wt. %. In some aspects, the toothpaste formulations include nanohydroxyapatite in an amount of about 10 wt. %, a combination of tea tree oil in an amount of about 1.0 wt. %, cinnamon oil in an amount of about 1.0 wt. % and coconut oil in an amount of about 1.0 wt. %, and the fluoride toothpaste in an amount of about 87 wt. %. In some aspects, the formulations may include nanohydroxyapatite in an amount of about 12 wt. %, a combination of tea tree oil in an amount of about 1.0 wt. %, cinnamon oil in an amount of about 1.0 wt. % and coconut oil in an amount of about 1.0 wt. %, and the fluoride toothpaste in an amount of about 85 wt. %.

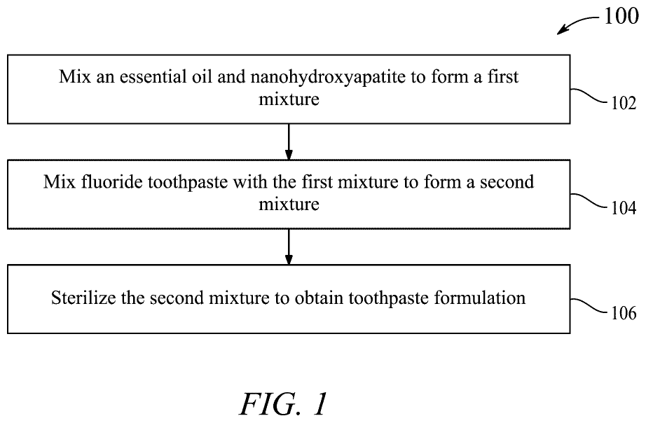

Referring to FIG. 1, a schematic flow chart of a method 100 of preparing toothpaste formulations is illustrated, according to an aspect of the present disclosure. The order in which the method 100 is described is not intended to be construed as a limitation, and any number of the described steps can be combined in any order to implement the process 100. Additionally, individual steps may be removed or skipped from the process 100 without departing from the spirit and scope of the present disclosure.

At step 102, the method 100 includes mixing an essential oil with a nanohydroxyapatite to form a first mixture. The mixing may be performed manually by a stirrer, and the solution may be constantly stirred while being mixed.

At step 104, the method 100 includes mixing the first mixture with a fluoride toothpaste to form a second mixture. The mixing is carried out such that a first portion of the essential oil is adsorbed onto, bonded to and/or dispersed in the nanohydroxyapatite or bonded to the nanohydroxyapatite. The other portion of the essential oil is dissolved in the fluoride toothpaste. Manual mixing is performed while preparing the second mixture to ensure there is no phase separation in the final mixture. In another embodiment the nanohydroxyapatite is formed in the presence of the essential oil thereby forming a nanohydroxyapatite having a portion of the essential oil dispersed therein.

Preferably the mass ratio of the mass of the first portion of the essential oil that is adsorbed onto, bonded to and/or dispersed in nanohydroxyapatite to the mass of the essential oil dissolved in the fluoride toothpaste is 5:1 to 1:5, preferably 3:1 to 1:3, 2:1 to 1:2 or about 1:1. Preferably an amount of at least 50wt. % of the total weight of the essential oil is dispersed in the nano-hydroxyapatite.

In one embodiment of the invention, at least a portion of the essential oil is dispersed in the nano-hydroxyapatite. At least partial dispersion of an essential oil in nano-hydroxyapatite occurs when the hydroxyapatite is formed in the presence of the activated oil. Preferably the nano-hydroxyapatite is made by wet chemical deposition or sol-gel precipitation. The reaction of calcium hydroxide with phosphoric acid in the presence of the essential oil, optionally further in the presence of an emulsifier such as CTAB, results in the precipitation of a nano-hydroxyapatite material that includes the essential oil in and on particles of the nano-hydroxyapatite. In a preferable embodiment of the invention, nano-hydroxyapatite particles consist of hydroxyapatite and a minor amount of the essential oil, for example, 0.1% by weight or less of the essential oil based on the total weight of the nano-hydroxyapatite, more preferably 0.05 wt. % by weight, 0.01 wt. %, or 0.001 wt. % or less of the essential oil.

At step 106, the method 100 includes sterilizing the second mixture to ensure the removal of contaminants or microorganisms if present in the formulation. The sterilization may be done by exposing the mixture to ultraviolet radiation for a specific time period which may vary from a few seconds to a few minutes. The resulting product is a smooth, homogenous antibacterial and re-mineralizing toothpaste formulation.

### EXAMPLES

The following examples demonstrate the toothpaste formulations and their assessments for their re-mineralizing and antibacterial properties as described herein. The examples are provided solely for illustration and are not to be construed as limitations of the present disclosure, as many variations thereof are possible without departing from the spirit and scope of the present disclosure.

### Example 1

### Formation of Three Different Experimental Compositions

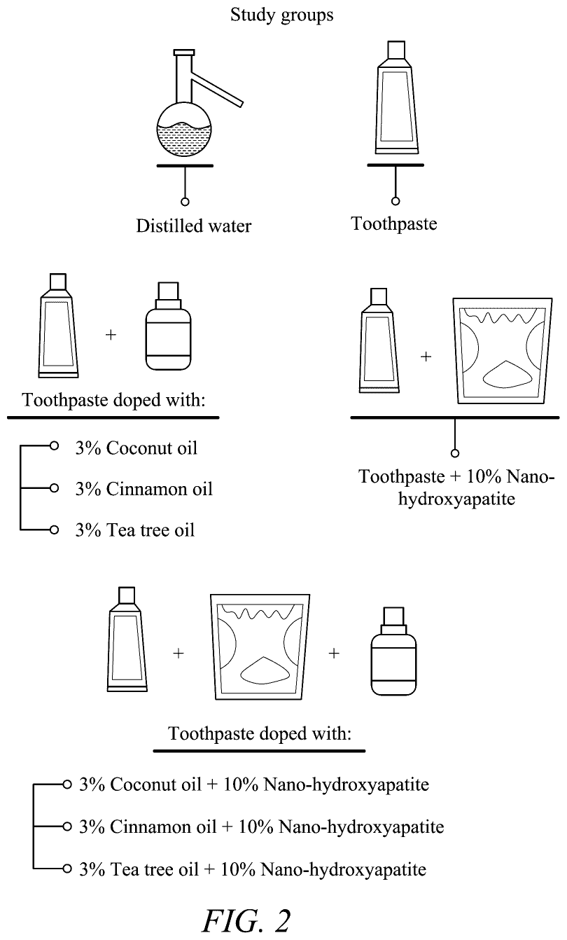

Three different experimental compositions were formed to assess the re-mineralizing and antibacterial properties of the toothpaste formulations, as illustrated in FIG. 2. The first group included toothpaste formulations having toothpaste doped with only essential oils. The second group included toothpaste formulations having toothpaste doped with nano-hydroxyapatite. The third group included different toothpaste formulations containing toothpastes doped with different essential oils and nano-hydroxyapatite.

### Example 2

### Remineralization Assessment

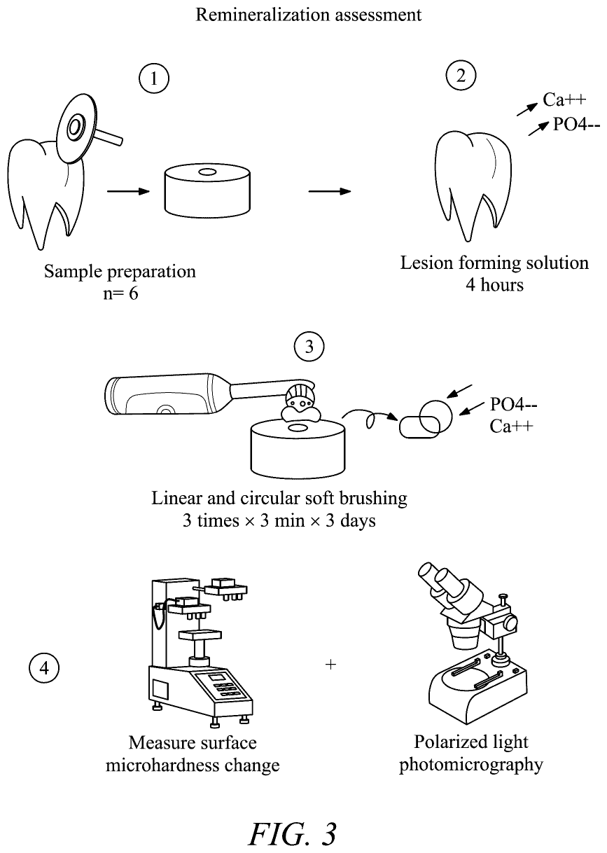

Samples were prepared as described in Example 1. Referring to FIG. 3, the experimental tooth was exposed to a lesion-forming solution for 4 hours. Once lesions were formed, the tooth was brushed with the experimental toothpastes including the toothpaste formulation of the present disclosure (fluoridated toothpaste), a fluoride toothpaste without the essential oils (positive control), n-hydroxyapatite (HA), and/or essential oils (coconut oil/cinnamon oil/tea tree oil), with linear and circular soft brushing 3 times a day for at least 3 days. The surface microhardness change was determined using polarized light photomicrography.

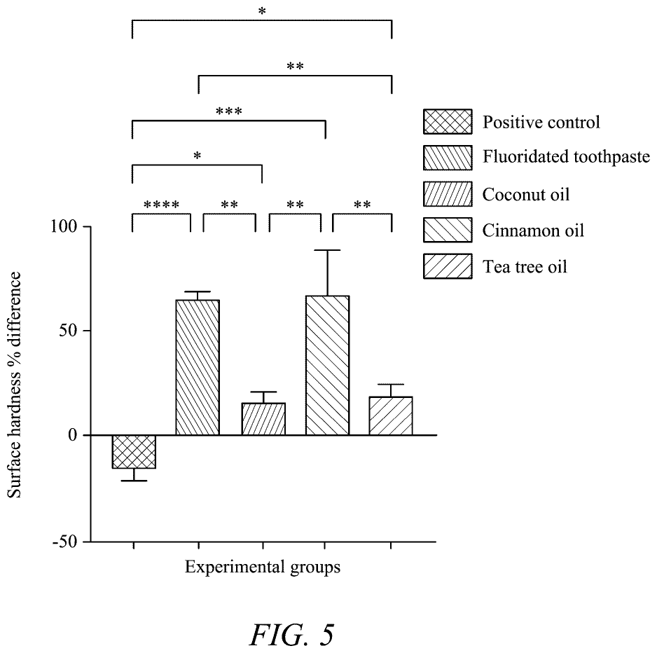

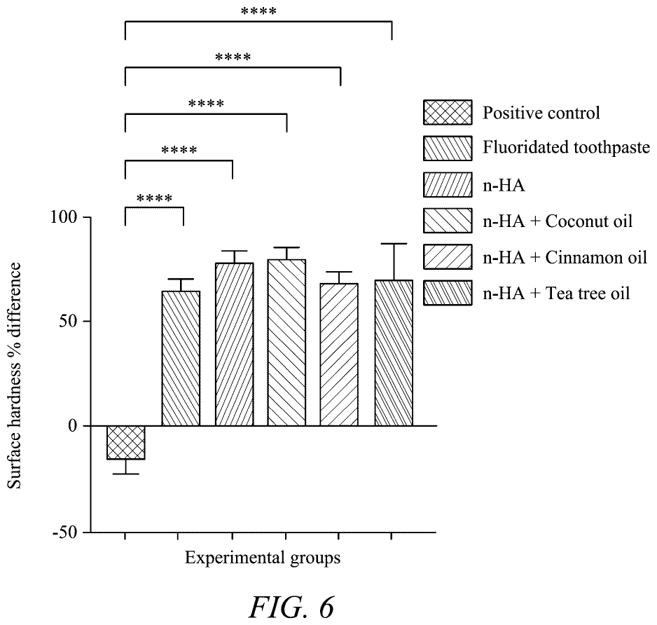

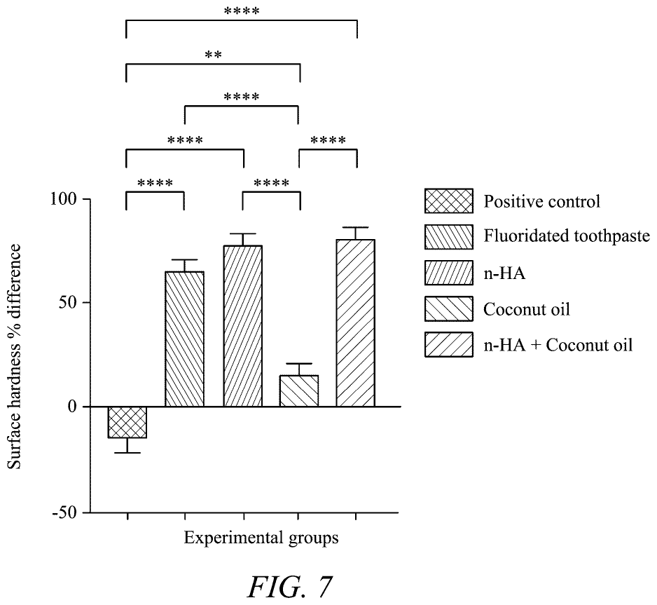

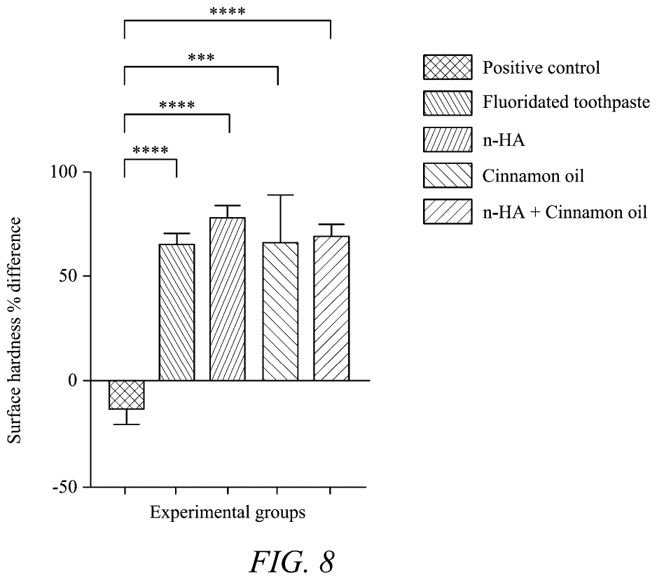

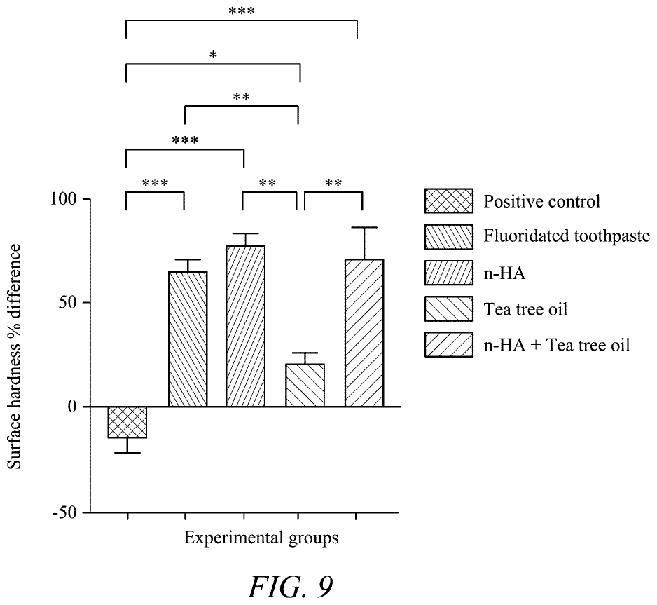

The results of this study are depicted in FIG. 5-FIG. 9. Surface microhardness is a physical property that measures the effect of chemical and physical agents on the hard tissues of teeth. This test can evaluate the resistance of enamel treated with the experimental toothpaste. The toothpaste formulation of the present disclosure demonstrates a significant increase in surface microhardness compared to the positive control, and comparable to the essential oils, as can be seen from FIG. 5 to FIG. 9, which indicate the increased resistance of the toothpaste formulation to enamel demineralization.

### Example 3

### Anti-Bacterial Property Assessment

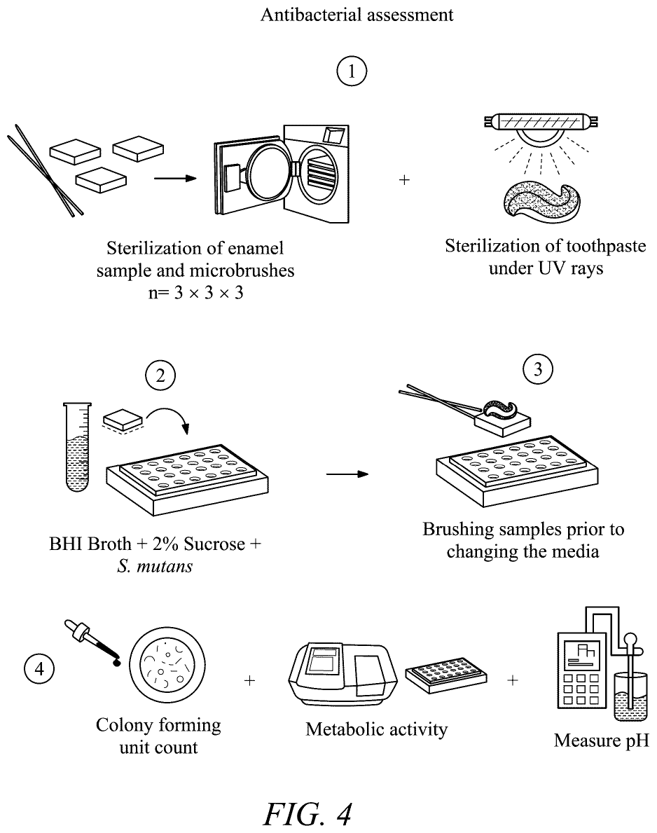

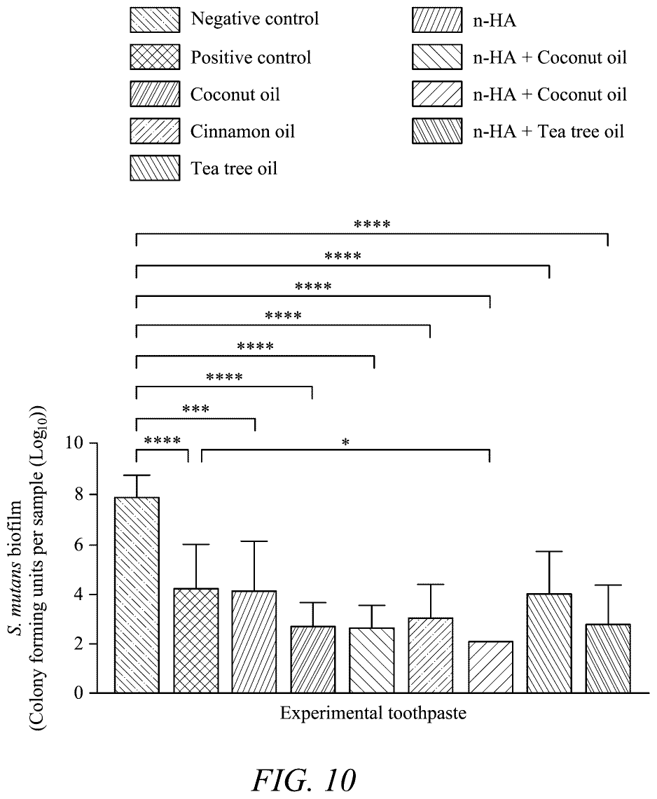

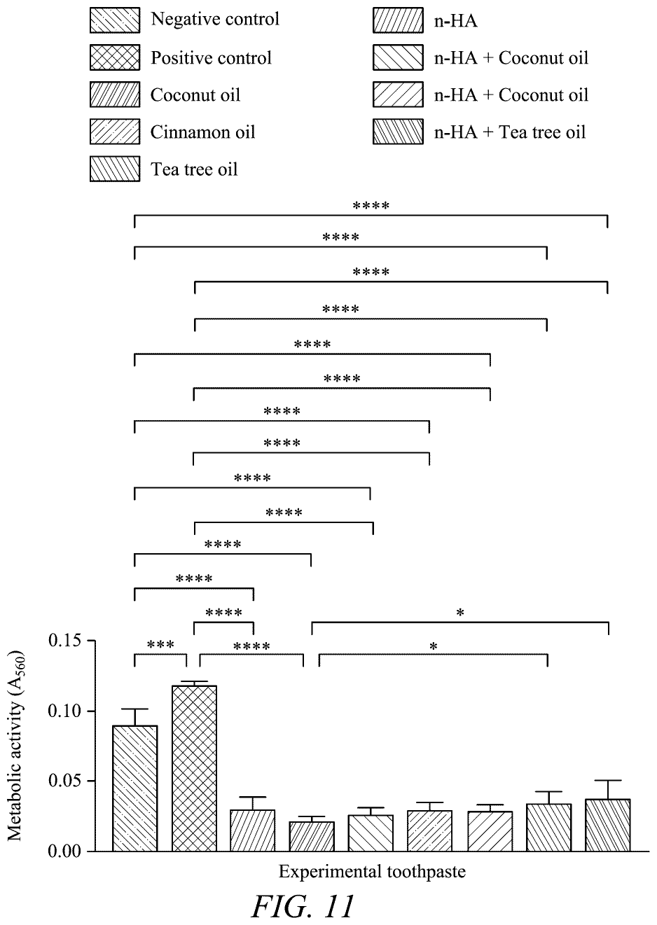

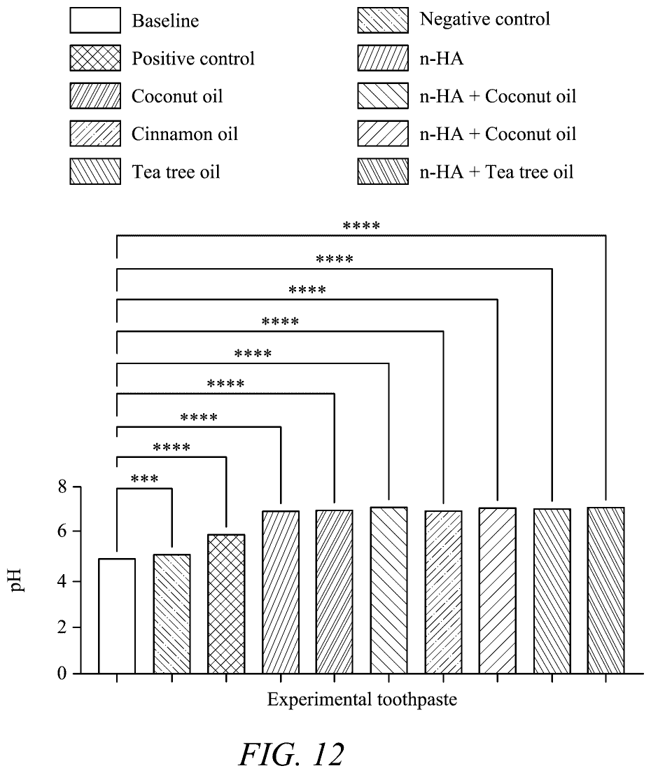

The antibacterial property assessment for all the experimental toothpaste was done by sterilizing the enamel samples and micro brushes and sterilizing the toothpaste under UV rays, as illustrated in FIG. 4. The colony-forming counts, metabolic activity, and pH were measured for all toothpaste formulations against the bacterium S. mutans. The antibacterial properties, namely, colony-forming counts, metabolic activity, and pH, of the experimental toothpaste after manual brushing are depicted in FIG. 10-FIG. 12, respectively.

Formulations incorporating 10% nano-hydroxyapatite and 3% essential oils and combinations thereof impart a detrimental biological effect on S. mutans biofilms by significantly reducing biofilm formation and metabolic activity.

A first embodiment describes the toothpaste formulation, including a nano-hydroxyapatite in an amount of about 8 wt. % to about 12 wt. %, an essential oil in an amount of about 2.8 wt. % to about 3.2 wt. %, wherein the essential oil is selected from the group consisting of tea tree oil, coconut oil, cinnamon oil and combinations thereof, and the balance fluoride toothpaste, wherein wt. % is based on a total weight of the toothpaste formulation. A first portion of the essential oil is dissolved in the fluoride toothpaste and a second portion of the essential oil is bonded or adsorbed in the nano-hydroxyapatite.

In an aspect, the nano-hydroxyapatite is present in an amount of about 10 wt. %, the essential oil is tea tree oil and is present in an amount of about 3 wt. %, and the fluoride toothpaste is present in an amount of about 87 wt. %.

In an aspect, the nano-hydroxyapatite is present in an amount of about 10 wt. %, the essential oil is coconut oil and is present in an amount of about 3 wt. %, and the fluoride toothpaste is present in an amount of about 87 wt. %.

In an aspect, the nano-hydroxyapatite is present in an amount of about 10 wt. %, the essential oil is cinnamon oil and is present in an amount of about 3 wt. %, and the fluoride toothpaste is present in an amount of about 87 wt. %.

In an aspect, the essential oil is a combination of tea tree oil present in an amount of about 1.5 wt. % and coconut oil present in an amount of about 1.5 wt. %, and the fluoride toothpaste is present in an amount in a range of about 85 wt. % to about 89 wt. %.

In an aspect, the essential oil is a combination of tea tree oil present in an amount of about 1.5 wt. % and cinnamon oil present in an amount of about 1.5 wt. %, and the fluoride toothpaste is present in an amount in a range of about 85 wt. % to about 89 wt. %.

In an aspect, the essential oil is a combination of cinnamon oil present in an amount of about 1.5 wt. % and coconut oil present in an amount of about 1.5 wt. %, and the fluoride toothpaste is present in an amount in a range of about 85 wt. % to about 89 wt. %.

In an aspect, the essential oil is a combination of tea tree oil present in an amount of about 1.0 wt. %, cinnamon oil present in an amount of about 1.0 wt. % and coconut oil present in an amount of about 1.0 wt. %, and the fluoride toothpaste is present in an amount in a range of about 85 wt. % to about 89 wt. %.

In an aspect, the nano-hydroxyapatite is present in an amount of about 8 wt. %, the essential oil is a combination of tea tree oil present in an amount of about 1.0 wt. %, cinnamon oil present in an amount of about 1.0 wt. % and coconut oil present in an amount of about 1.0 wt. %, and the fluoride toothpaste is present in an amount of about 89 wt. %.

In an aspect, the nano-hydroxyapatite is present in an amount of about 10 wt. %, the essential oil is a combination of tea tree oil present in an amount of about 1.0 wt. %, cinnamon oil present in an amount of about 1.0 wt. % and coconut oil present in an amount of about 1.0 wt. %, and the fluoride toothpaste is present in an amount of about 87 wt. %.

In an aspect, the nano-hydroxyapatite is present in an amount of about 12 wt. %, the essential oil is a combination of tea tree oil present in an amount of about 1.0 wt. %, cinnamon oil present in an amount of about 1.0 wt. % and coconut oil present in an amount of about 1.0 wt. %, and the fluoride toothpaste is present in an amount of about 85 wt. %.

In an aspect, the fluoride toothpaste includes sodium fluoride in an amount in a range of about 0.15 wt. %.

A second embodiment describes the toothpaste formulation consisting of a nano-hydroxyapatite in an amount of about 8 wt. % to about 12 wt. %, an essential oil in an amount of about 2.8 wt. % to about 3.2 wt. %, wherein the essential oil is selected from the group consisting of tea tree oil, coconut oil, cinnamon oil and combinations thereof, and the balance fluoride toothpaste, wherein wt. % is based on a total weight of the toothpaste formulation. A first portion of the essential oil is dissolved in the fluoride toothpaste and a second portion of the essential oil is bonded or adsorbed in the nano-hydroxyapatite.

In an aspect, the essential oil is a combination of tea tree oil present in an amount of about 1.0 wt. %, cinnamon oil present in an amount of about 1.0 wt. % and coconut oil present in an amount of about 1.0 wt. %, and the fluoride toothpaste is present in an amount in a range of about 85 wt. % to about 89 wt. %.

In an aspect, the nano-hydroxyapatite is present in an amount of about 10 wt. %, the essential oil is tea tree oil and present in an amount of about 3 wt. %, and the fluoride toothpaste is present in an amount of about 87 wt. %.

In an aspect, the nano-hydroxyapatite is present in an amount of about 10 wt. %, and the essential oil is coconut oil and present in an amount of about 3 wt. %, and the fluoride toothpaste is present in an amount of about 87 wt. %.

In an aspect, the nano-hydroxyapatite is present in an amount of about 10 wt. %, and the essential oil is cinnamon oil and present in an amount of about 3 wt. %, and the fluoride toothpaste is present in an amount of about 87 wt. %.

In an aspect, the essential oil is a combination of tea tree oil present in an amount of about 1.5 wt. % and coconut oil present in an amount of about 1.5 wt. %, and the fluoride toothpaste is present in an amount in a range of about 85 wt. % to about 89 wt. %.

In an aspect, the essential oil is a combination of tea tree oil present in an amount of about 1.5 wt. % and cinnamon oil present in an amount of about 1.5 wt. %, and the fluoride toothpaste is present in an amount in a range of about 85 wt. % to about 89 wt. %.

A third embodiment describes a method 100 for preparing the toothpaste formulation. The method 100 includes mixing an essential oil in an amount of about 2.8 wt. % to about 3.2 wt. %, wherein the essential oil is selected from the group consisting of tea tree oil, coconut oil, cinnamon oil and combinations thereof, with a nano-hydroxyapatite in an amount of about 8 wt. % to about 12 wt. % to form a first mixture, and mixing a fluoride toothpaste in an amount of about 85 wt. % to about 89 wt. % into the first mixture to form a second mixture, wherein a first portion of the essential oil is dissolved in the fluoride toothpaste and a second portion of the essential oil is bonded to, adsorbed in and/or dispersed in the nano-hydroxyapatite, and sterilizing the second mixture under ultraviolet lighting.

Numerous modifications and variations of the present disclosure are possible in light of the above teachings. It is therefore to be understood that within the scope of the appended claims, the invention may be practiced otherwise than as specifically described herein.

1. A toothpaste formulation, comprising:
a nano-hydroxyapatite in an amount of about 8 wt. % to about 12 wt. %,
an essential oil in an amount of about 2.8 wt. % to about 3.2 wt. %, wherein the essential oil is selected from the group consisting of tea tree oil, coconut oil, cinnamon oil and combinations thereof, and
the balance fluoride toothpaste, wherein wt. % is based on a total weight of the toothpaste formulation,
wherein a first portion of the essential oil is dissolved in the fluoride toothpaste and a second portion of the essential oil is bonded to the nano-hydroxyapatite, adsorbed on the surface of the nano-hydroxyapatite and/or dispersed in the nano-hydroxyapatite.

2. The toothpaste formulation of claim 1, wherein:
the nano-hydroxyapatite is present in an amount of about 10 wt. %,
the essential oil is tea tree oil present in an amount of about 3 wt. % and is dispersed in the nano-hydroxyapatite, and
the fluoride toothpaste is present in an amount of about 87 wt. %.

3. The toothpaste formulation of claim 1, wherein:
the nano-hydroxyapatite is present in an amount of about 10 wt. %, and
the essential oil is coconut oil present in an amount of about 3 wt. % and is dispersed in the nano-hydroxyapatite, and
the fluoride toothpaste is present in an amount of about 87 wt. %.

4. The toothpaste formulation of claim 1, wherein:
the nano-hydroxyapatite is present in an amount of about 10 wt. %, and
the essential oil is cinnamon oil present in an amount of about 3 wt. % and is dispersed in the nano-hydroxyapatite, and
the fluoride toothpaste is present in an amount of about 87 wt. %.

5. The toothpaste formulation of claim 1, wherein:
the essential oil is a combination of tea tree oil present in an amount of about 1.5 wt. % and coconut oil present in an amount of about 1.5 wt. %, wherein the combination is dispersed in the nano-hydroxyapatite, and
the fluoride toothpaste is present in an amount in a range of about 85 wt. % to about 89 wt. %.

6. The toothpaste formulation of claim 1, wherein:
the essential oil is a combination of tea tree oil present in an amount of about 1.5 wt. % and cinnamon oil present in an amount of about 1.5 wt. %, and
the fluoride toothpaste is present in an amount in a range of about 85 wt. % to about 89 wt. %.

7. The toothpaste formulation of claim 1, wherein:
the essential oil is a combination of cinnamon oil present in an amount of about 1.5 wt. % and coconut oil present in an amount of about 1.5 wt. %, and
the fluoride toothpaste is present in an amount in a range of about 85 wt. % to about 89 wt. %.

8. The toothpaste formulation of claim 1, wherein:
the essential oil is a combination of tea tree oil present in an amount of about 1.0 wt. %, cinnamon oil present in an amount of about 1.0 wt. % and coconut oil present in an amount of about 1.0 wt. %, and
the fluoride toothpaste is present in an amount in a range of about 85 wt. % to about 89 wt. %.

9. The toothpaste formulation of claim 1, wherein:
the nano-hydroxyapatite is present in an amount of about 8 wt. %,
the essential oil is a combination of tea tree oil present in an amount of about 1.0 wt. %, cinnamon oil present in an amount of about 1.0 wt. % and coconut oil present in an amount of about 1.0 wt. %, and
the fluoride toothpaste is present in an amount of about 89 wt. %.

10. The toothpaste formulation of claim 1, wherein:
the nano-hydroxyapatite is present in an amount of about 10 wt. %,
the essential oil is a combination of tea tree oil present in an amount of about 1.0 wt. %, cinnamon oil present in an amount of about 1.0 wt. % and coconut oil present in an amount of about 1.0 wt. %, and
the fluoride toothpaste is present in an amount of about 87 wt. %.

11. The toothpaste formulation of claim 1, wherein:
the nano-hydroxyapatite is present in an amount of about 12 wt. %,
the essential oil is a combination of tea tree oil present in an amount of about 1.0 wt. %, cinnamon oil present in an amount of about 1.0 wt. % and coconut oil present in an amount of about 1.0 wt. %, and
the fluoride toothpaste is present in an amount of about 85 wt. %.

12. The toothpaste formulation of claim 1, wherein the fluoride toothpaste includes sodium fluoride in an amount in a range of about 0.15 wt. %.

13. A toothpaste formulation, consisting of:
a nano-hydroxyapatite in an amount of about 8 wt. % to about 12 wt. %,
an essential oil in an amount of about 2.8 wt. % to about 3.2 wt. %, wherein the essential oil is selected from the group consisting of tea tree oil, coconut oil, cinnamon oil and combinations thereof, and
the balance fluoride toothpaste, wherein wt. % is based on a total weight of the toothpaste formulation,
wherein a first portion of the essential oil is dissolved in the fluoride toothpaste and a second portion of the essential oil is bonded or adsorbed in the nano-hydroxyapatite.

14. The toothpaste formulation of claim 13, wherein:
the essential oil is a combination of tea tree oil present in an amount of about 1.0 wt. %, cinnamon oil present in an amount of about 1.0 wt. % and coconut oil present in an amount of about 1.0 wt. %, and
the fluoride toothpaste is present in an amount in a range of about 85 wt. % to about 89 wt. %.

15. The toothpaste formulation of claim 13, wherein:
the nano-hydroxyapatite is present in an amount of about 10 wt. %,
the essential oil is tea tree oil present in an amount of about 3 wt. %, and
the fluoride toothpaste is present in an amount of about 87 wt. %.

16. The toothpaste formulation of claim 13, wherein:
the nano-hydroxyapatite is present in an amount of about 10 wt. %, and
the essential oil is coconut oil present in an amount of about 3 wt. %, and
the fluoride toothpaste is present in an amount of about 87 wt. %.

17. The toothpaste formulation of claim 13, wherein:
the nano-hydroxyapatite is present in an amount of about 10 wt. %, and
the essential oil is cinnamon oil present in an amount of about 3 wt. %, and
the fluoride toothpaste is present in an amount of about 87 wt. %.

18. The toothpaste formulation of claim 13, wherein:
the essential oil is a combination of tea tree oil present in an amount of about 1.5 wt. % and coconut oil present in an amount of about 1.5 wt. %, and
the fluoride toothpaste is present in an amount in a range of about 85 wt. % to about 89 wt. %.

19. The toothpaste formulation of claim 1, wherein:
the essential oil is a combination of tea tree oil present in an amount of about 1.5 wt. % and cinnamon oil present in an amount of about 1.5 wt. %, and
the fluoride toothpaste is present in an amount in a range of about 85 wt. % to about 89 wt. %.

20. A method of formulating an antibiotic, remineralizing toothpaste, comprising:
mixing an essential oil in an amount of about 2.8 wt. % to about 3.2 wt. %, wherein the essential oil is selected from the group consisting of tea tree oil, coconut oil, cinnamon oil and combinations thereof, with a nano-hydroxyapatite in an amount of about 8 wt. % to about 12 wt. % to form a first mixture, and
mixing a fluoride toothpaste in an amount of about 85 wt. % to about 89 wt. % into the first mixture to form a second mixture, wherein a first portion of the essential oil is dissolved in the fluoride toothpaste and a second portion of the essential oil is bonded or adsorbed in the nano-hydroxyapatite, and
sterilizing the second mixture under ultraviolet lighting.
